(19) United States
(12) Patent Application Publication
(10) Pub. No.: US 2014/0316401 A1
(43) Pub. Date: Oct. 23, 2014

(54) SHEATH ASSEMBLIES FOR ELECTROSURGICAL INSTRUMENTS

(71) Applicant: INTUITIVE SURGICAL OPERATIONS, INC., Sunnyvale, CA (US)

(72) Inventors: Samuel T. CREWS, Palomar Park, CA (US); Craig R. GERBI, Half Moon Bay, CA (US)

(73) Assignee: INTUITIVE SURGICAL OPERATIONS, INC., Sunnyvale, CA (US)

(21) Appl. No.: 14/204,493

(22) Filed: Mar. 11, 2014

Related U.S. Application Data
(60) Provisional application No. 61/783,813, filed on Mar. 14, 2013.

(51) Int. Cl. A61B 18/14; A61B 18/12; A61B 19/00

(52) U.S. Cl. CPC A61B 18/1445; A61B 19/2203; A61B 18/1206; A61B 2018/00577
USPC 606/34

(57) ABSTRACT

An electrosurgical device may include an elongated shaft having a distal end and a proximal end, an electrosurgical end effector coupled to the distal end of the elongated shaft, an electrically insulative sheath disposed around at least a proximal end portion of the end effector, and an electrically insulative viscous material disposed to provide a barrier to liquid entry into an interior region defined by the sheath.

### REFERENCE TO RELATED APPLICATIONS

This application claims priority to U.S. Provisional Application No. 61/783,813, filed Mar. 14, 2013, which is incorporated by reference herein in its entirety.

### TECHNICAL FIELD

The present teachings relate to devices, systems, and methods for inhibiting or preventing conduction of electrical current from an electrosurgical instrument along undesirable paths, including to unwanted locations of a patient and/or between components of the instrument itself. In particular, the present teachings relate to sheath assemblies for use with electrosurgical instruments.

Various electrosurgical instruments, which generally use high-frequency alternating current, perform a procedure on tissue of an organism, e.g., a human patient, using heat produced by electrical energy (e.g. cautery energy) applied to the tissue. Such instruments typically include an end effector disposed at a distal end of an instrument shaft. Electrosurgical instruments may include, for example, monopolar instruments or bipolar instruments. Monopolar instruments typically deliver electrical energy through a single source (e.g., positive pole) electrode. A return (e.g., negative pole), or sink, electrode returns electrical energy back to an energy generator disposed externally to the patient. Thus, monopolar electrosurgical instruments form a complete electrical circuit from the single active electrode, to the target tissue, to the return electrode (typically in contact with the patient being treated), and back to the electrical energy supply source (e.g., electrical energy generator) that is electrically coupled to the electrosurgical instrument. Monopolar electrosurgical instruments can have end effectors including, but not limited to, for example, hooks, spatulas, shears/scissors including two blades energized with the same electric potential, cautery probes, irrigators, etc.

Bipolar electrosurgical instruments typically deliver electrical energy through two electrodes (e.g., source and sink electrodes) separately, and the return path for the current is from one electrode through the other electrode. Current travels from the source (e.g., positive pole) electrode to the sink (e.g., negative pole) electrode. The electrodes of bipoloar electrosurgical instruments are typically disposed at two jaws of the end effector of the electrosurgical instrument. Examples of bipolar instrument end effectors include, but are not limited to, for example, graspers, forceps, clamps, etc., which are generally used for sealing vessels and vascular tissue, grasping vessels, and/or cauterizing or coagulating tissue, and other similar surgical procedures.

Thus, the end effectors of electrosurgical instruments can be used to perform a variety of procedures, such as, for example, incision, sealing, coagulation, ablation, and the like in minimally invasive procedures, either performed manually or via teleoperated (also referred to as robotic) surgical systems. In some cases, surgeons work through incisions and manipulate such electrosurgical instruments through a cannula. Electrosurgical instruments may be used in both manual minimally invasive surgical systems or in automated, teleoperated (robotic) surgical systems, such as, for example the da Vinci® system commercialized by Intuitive Surgical, Inc. of Sunnyvale, Calif.

The electrosurgical instrument end effector can include a variety of components formed from electrically conductive materials, such as, for example, metal (e.g., stainless steel, and the like). Further, to provide the end effector with a range of motion, some electrosurgical instruments include a mechanical wrist structure that is used to support the end effector at the distal end of the instrument shaft. Such a wrist structure or support mechanism can be made of a variety of materials that may be electrically conductive, including metal (e.g., stainless steel and like) materials. Wrist structures are operational in a wet environment, and as mentioned above, can be coupled to the electrocautery end effector in order to enhance maneuverability and positioning of the end effector.

Due to the use of electrical elements and conduction of electricity through various portions of the electrosurgical instrument, a sheath made of an electrically insulative material may be used. In some cases, the sheath can be slid on over the end effector of the instrument and generally disposed over various electrically conductive components associated with the end effector. Such a sheath can be configured and positioned to inhibit conduction of electrical current from the electrically live components to the patient, thus preventing unwanted electrically-related patient burns at a location away from the electrocautery end effector, for example, including at an area proximate the wrist member. However, in some cases the wall thickness of the sheath can add to the overall instrument diameter, which can pose challenges in minimally invasive applications where smaller overall instrument sizes are desirable. In some cases, a sheath is placed over the electrically live wrist and a proximal end portion of the end effector, and the sheath has an outer diameter such that when positioned on the instrument the sheath outer diameter is generally the same as that of the shaft of the electrosurgical instrument. The sheath can be permanent or removable and potentially reusable (e.g., after sterilization).

Insulative sheaths that are intended to be slid over the end effector and into position may nevertheless allow for blood, saline, and other materials in the wet environment of the surgical application to enter into interstitial spaces between the sheath and the instrument, and in the interior of the instrument components, e.g., in particular the associated end effector components. This can cause the potential for the formation of unintended electrical pathways, which can cause short-circuit pathways, during use for an electrosurgical procedure.

Accordingly, there continues to exist a need to provide effective electrical insulation to components associated with end effectors of electrosurgical instruments to inhibit or prevent conduction of electrical current along undesirable pathways, for example, to undesired locations of the patient and/or that include parts of the electrosurgical instrument component. There also exists a need to maintain the overall size of electrosurgical instruments relatively small for various applications, while also including sufficient electrically insulative material at locations associated with the end effector, support structures associated with the end effector, and/or distal end portions of the instrument shaft where it is desirable to inhibit and/or prevent conduction of electrical current.

### SUMMARY

The present teachings may solve one or more of the above-mentioned problems and/or may demonstrate one or more of the above-mentioned desirable features. Other features and/or advantages may become apparent from the description that follows.

In one exemplary embodiment, the present disclosure contemplates an electrosurgical device that includes an elongated shaft having a distal end and a proximal end, an electrosurgical end effector coupled to the distal end of the elongated shaft, an electrically insulative sheath disposed around at least a proximal end portion of the end effector, and an electrically insulative viscous material disposed to provide a barrier to liquid entry into an interior region defined by the sheath.

In another exemplary embodiment, the present disclosure contemplates a sheath assembly for an electrosurgical instrument. The sheath assembly can include an electrically insulative sheath configured to be positioned on a surgical instrument to surround at least a proximal end of an end effector of the surgical instrument, and an electrically insulative viscous material disposed within an interior region defined by the sheath.

In yet another exemplary embodiment, the present disclosure contemplates an electrosurgical device that includes an electrosurgical instrument with an elongated shaft having a distal end and a proximal end, and an electrosurgical end effector coupled to the distal end of the elongated shaft. The device further can include an electrically insulative viscous material disposed in an amount and arrangement sufficient to protect against an unintended electrical pathway formed at least in part by a component of the electrosurgical instrument.

In another exemplary embodiment, the present disclosure contemplates a kit that includes an electrically insulative sheath configured to be positioned on a surgical instrument to surround at least a proximal end of an end effector of the surgical instrument, and an electrically insulative viscous material for application between the sheath and a surgical instrument in a position of the sheath on the surgical instrument, wherein the electrically insulative material has a viscosity sufficient to hold the material within an interior region defined by the sheath.

According to yet another exemplary embodiment, the present disclosure contemplates a method that includes applying an electrically insulative viscous material to one of a portion of an electrosurgical instrument that includes at least a proximal end region of an end effector of the electrosurgical instrument and an interior surface portion of a sheath, positioning a sheath on the electrosurgical instrument such that the electrically insulative viscous material is within an interior region defined by the sheath, and heat shrinking the sheath positioned on the electrosurgical instrument.

Additional aspects and advantages will be set forth in part in the description which follows, and in part will be obvious from the description, or may be learned by practice of the present teachings. The objects and advantages may be realized and attained by means of the elements and combinations particularly pointed out in the appended claims and their equivalents.

It is to be understood that both the foregoing general description and the following detailed description are exemplary and explanatory only and are not restrictive of the claims.

### BRIEF DESCRIPTION OF DRAWINGS

The teachings of the present invention can be readily understood by considering the following detailed description in conjunction with the accompanying drawings.

FIGS. 1A-1C are front elevation, diagrammatic views of an exemplary patient side cart, surgeon's console, and auxiliary control/vision cart, respectively, in a teleoperated surgical system;

FIGS. 2A and 2B are schematic views of an electrosurgical instrument in accordance with exemplary embodiments;

FIG. 3A is a partial side perspective view of an electrosurgical instrument in accordance with an exemplary embodiment;

FIG. 3B is a partial, top view of an end effector of an electrosurgical instrument depicted in partial cutaway view to illustrate interior parts of the instrument proximate the end effector;

FIG. 3C is the end effector of FIG. 3A with a sheath assembly in position in accordance with an exemplary embodiment; and

FIG. 4 is a cross-sectional view taken through line 4-4 in FIG. 3B.

### DETAILED DESCRIPTION OF EXEMPLARY EMBODIMENTS

Reference will now be made in detail to various exemplary embodiments of the present disclosure, examples of which are illustrated in the accompanying drawings. One skilled in the art would readily recognize from the following description that alternative embodiments exist without departing from the general principles of the present disclosure. This description and the accompanying drawings illustrate exemplary embodiments and should not be taken as limiting, with the claims defining the scope of the present teachings. Various mechanical, compositional, structural, electrical, and operational changes may be made without departing from the scope of this description and the invention as claimed, including equivalents.

In some instances, well-known structures, and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect actual shapes, sizes, or dimensions, for example, of electrosurgical instruments and their components.

For the purposes of this specification and appended claims, unless otherwise indicated, all numbers expressing quantities, percentages, or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

The terms “proximal” and “distal” are relative terms, where the term “distal” refers to the portion of the object furthest from an operator of the instrument and closest to the surgical site, such as the opening of the tool cover or the end effector of the instrument. The term “proximal” indicates the relative proximity to the operator of the surgical instrument and refers to the portion of the object closest to the operator and furthest from the surgical site. In this application, an end effector refers to a tool installed at the distal end of an instrument, including but not limited to forceps or graspers, needle drivers, scalpels, scissors, spatulas, blades, and other cauterizing tools.

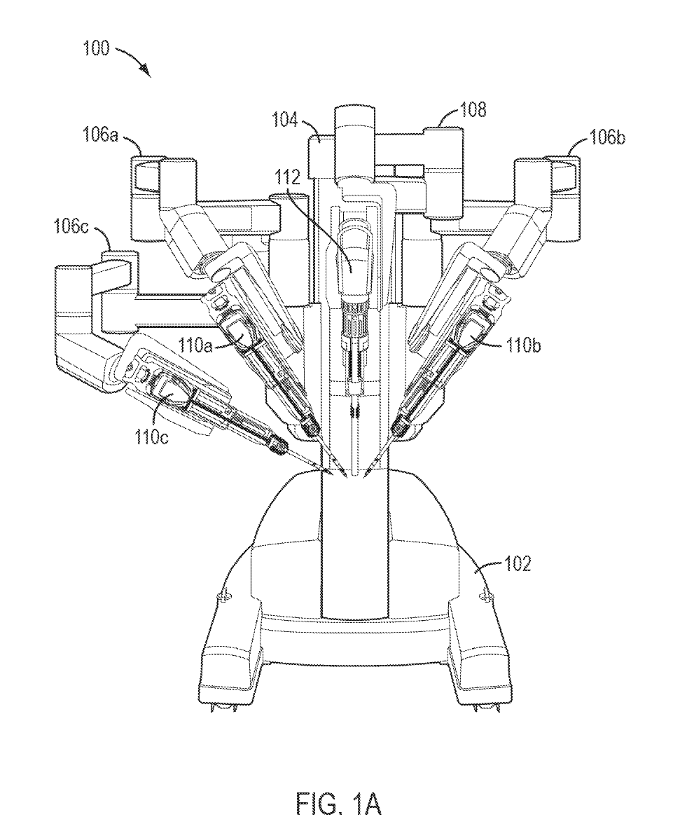

FIGS. 1A, 1B, and 1C are front elevation views of three exemplary embodiments of main components of a teleoperated (robotic) surgical system for minimally invasive surgery. These three components are interconnected so as to allow a surgeon, for example, with the assistance of a surgical team, to perform diagnostic and corrective surgical procedures on a patient. In an exemplary embodiment, a teleoperated surgical system in accordance with the present disclosure may be embodied as a da Vinci® surgical system commercialized by Intuitive Surgical, Inc. of Sunnyvale, Calif. Also, for a further explanation of a teleoperated surgical system, including a patient side cart, surgeon's console, and auxiliary control/vision cart, with which the present disclosure may be implemented, reference is made to U.S. Patent App. Pub. No. 2011/0071542 A1 (published Mar. 24, 2011), entitled “CURVED CANNULA SURGICAL SYSTEM,” which is incorporated by reference in its entirety herein. However, the present disclosure is not limited to any particular teleoperated surgical system, and one having ordinary skill in the art would appreciate that the disclosure herein may be applied in a variety of surgical applications, including other teleoperated surgical systems, as well as in manual surgical applications, such as, for example, laparoscopic and thoracoscopic procedures.

FIG. 1A is a front elevation view of an exemplary embodiment of a patient side cart 100 of a teleoperated surgical system. The patient side cart 100 includes a base 102 that rests on the floor, a support tower 104 mounted on the base 102, and one or more arms mounted on the support tower 104 and that support surgical instruments and/or vision instruments (e.g., a stereoscopic endoscope). As shown in FIG. 1A, arms 106a,106b are instrument arms that support and move the surgical instruments used to manipulate tissue, and arm 108 is a camera arm that supports and moves the endoscope. FIG. 1A also shows a third instrument arm 106c that is supported on the back side of support tower 104 and that is positionable to either the left or right side of the patient side cart as desired to conduct a surgical procedure. Interchangeable surgical instruments 110a,110b,110c can be installed on the instrument arms 106a,106b,106c, and an endoscope 112 can be installed on the camera arm 108. Those of ordinary skill in the art will appreciate that the arms that support the instruments and the camera may also be supported by a base platform (fixed or moveable) mounted to a ceiling or wall, or in some instances to another piece of equipment in the operating room (e.g., the operating table). Likewise, they will appreciate that two or more separate bases may be used (e.g., one base supporting each arm).

FIG. 1B is a front elevation view of an exemplary surgeon's console 120 of a teleoperated surgical system. The surgeon's console is equipped with left and right multiple degree-of-freedom (DOE) master tool manipulators (MTM's) 122a,122b, which are kinematic chains that are used to control the surgical tools (which include the endoscope and various cannulas mounted on arms 106, 108 of the patient side cart 100). The surgeon grasps a pincher assembly 124a,124b on each MTM 122a, 122b, typically with the thumb and forefinger, and can move the pincher assembly to various positions and orientations. When a tool control mode is selected, each MTM 122 is coupled to control a corresponding instrument arm 106 for the patient side cart 100, as those of ordinary skill in the art are familiar with. In some instances, control assignments between MTM's 122a,122b and arm 106a/instrument 110a combination and arm 106b/instrument 110b combination may also be exchanged.

The pincher assembly is typically used to operate a surgical end effector (e.g., scissors, grasping retractor, needle driver, hook, forceps, spatula, etc.) at the distal end of an instrument 110. For example, as those of ordinary skill in the art are familiar with, inputs at the pincher assemblies 124a, 124b can be coupled to control drive members at actuation interfaces at the arms 106. The drive members can in turn be coupled to and actuate various force transmission mechanisms disposed in proximally disposed transmission housings of the surgical instruments 110, which forces are transmitted along the instrument shafts to control movement of the instrument shaft, wrist (if any), and end effectors. The endoscope camera instrument is similarly controlled as those having ordinary skill in the art would appreciate.

Surgeon's console 120 also can include an image display system 126. In an exemplary embodiment, the image display is a stereoscopic display wherein left side and right side images captured by the stereoscopic endoscope 112 are output on corresponding left and right displays, which the surgeon perceives as a three-dimensional image on display system 126.

The surgeon's console 120 is typically located in the same operating room as the patient side cart 100, although it is positioned so that the surgeon operating the console is outside the sterile field. One or more assistants typically assist the surgeon by working within the sterile surgical field (e.g., to change tools on the patient side cart, to perform manual retraction, etc.). Accordingly, the surgeon operates remote from the sterile field, and so the console may be located in a separate room or building from the operating room. In some implementations, two consoles 120 (either co-located or remote from one another) may be networked together so that two surgeons can simultaneously view and control tools at the surgical site.

FIG. 1C is a front elevation view of an exemplary auxiliary control/vision cart 140 of the teleoperated surgical system. The cart 140 houses the surgical system's central electronic data processing unit 142 and vision equipment 144. The central electronic data processing unit includes much of the data processing used to operate the surgical system. In various other implementations, however, the electronic data processing also may be distributed in the surgeon console and patient side cart. The vision equipment includes camera control units for the left and right image capture functions of the stereoscopic endoscope 112. The vision equipment also includes illumination equipment (e.g., Xenon lamp) that provides illumination for imaging the surgical site. As shown in FIG. 1C, the auxiliary control/vision cart 140 includes an optional display 146 (e.g., a touchscreen monitor), which may be mounted elsewhere, such as on the patient side cart 100. The auxiliary control/vision cart 140 further includes space 148 for optional auxiliary surgical equipment, such as electrosurgical units, insufflators, and/or other flux supply and control units. The patient side cart 100 (FIG. 1A) and the surgeon's console 120 (FIG. 18) are coupled via optical fiber communications links to the auxiliary control/vision cart 140 so that the three components together act as a single teleoperated minimally invasive surgical system that provides an intuitive telepresence for the surgeon. As mentioned above, a second surgeon's console may be included so that a second surgeon can, e.g., proctor the first surgeon's work.

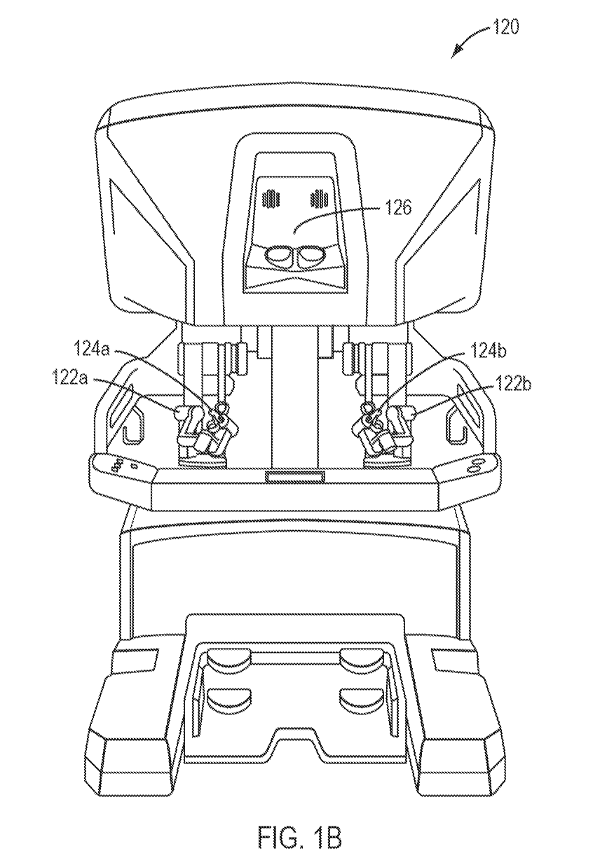

FIGS. 2A and 28 are perspective and interior schematic views of an exemplary embodiment of an electrosurgical instrument 200. The electrosurgical instrument 200 includes a main instrument shaft 220 that has a transmission housing 210 disposed at a proximal end of the shaft 220 and an end effector 240 disposed at a distal end of the shaft 220 (with the distal and proximal directions being labeled in FIG. 28). The shaft 220 may be a relatively flexible structure that can bend and curve, or can be a relatively rigid structure that does not bend significantly to follow curved paths. Optionally, the instrument 200 also can include a multi-DOF articulatable wrist structure 230 that supports the end effector 240 and permits multi-DOF movement of the end effector in arbitrary pitch and yaw. Those having ordinary skill in the art are familiar with a variety of wrist structures used to permit multi-DOF movement of a surgical instrument end effector. In various exemplary embodiments, however, the wrist structure may be eliminated (e.g., as shown in FIG. 28 for simplicity) without departing from the scope of the present disclosure. In general, the end effector 240 has a support clevis 222 that supports and mounts the end effector 240 relative to the instrument shaft 220 or wrist structure 230, if any.

Control of the end effector 240 and optional wrist structure 230 may be accomplished using force transmission members 226 that transmit forces from various drive mechanisms (not shown) in the transmission housing 210 attached to an actuation interface assembly of a patient side cart (e.g., as shown in FIG. 1A), as those of ordinary skill in the art are familiar with. The force transmission members 226 can have a variety of forms and be implemented in various ways, exemplary arrangements and operations of which are disclosed, for example, in U.S. Pat. App. Pub. No. 2011/0071542 A1 (published Mar. 24, 2011), entitled “CURVED CANNULA SURGICAL SYSTEM,” incorporated by reference herein. In various exemplary embodiments, an electrically insulative sheath (not shown in FIG. 28) can cover at least the wrist structure 230, if any, the clevis portion 222, and a proximal end portion of the end effector 240. If the remainder of the main shaft 220 does not receive an electrical charge and is not electrically conductive, it may be unnecessary to cover the entire shaft with an electrically insulative sheath. However, in alternative embodiments, the sheath may extend to cover the entire length or a portion of the main shaft 220.

The electrically insulative sheaths in accordance with various exemplary embodiments of the present disclosure can be made of one or more materials and have an overall configuration that makes them impact and tear resistant, substantially electrically non-conductive, tolerant to high temperatures, and sufficiently elastic so as to avoid significantly impeding the movement of the end effector, (e.g., including jaws and/or blades of shears), or other components that exhibit a relatively large relative motion, wrist structure, and/or distal portion of the shaft. Further, at least in some applications, it is desirable to provide a sheath structure having a wall thickness that is as small as possible to minimize the outer dimensions of the instrument with the sheath disposed thereon.

Regarding the latter, various exemplary embodiments utilize a tube made of a heat-shrinkable material as the sheath, with the tube being heat shrunk around at least the proximal portion of the end effector and support structure (including, for example a wrist structure if any) that supports the end effector to the instrument shaft. A sheath of heat-shrinkable material can provide sufficient electrical insulation; be tightly secured in a conforming manner to the contours of the instrument, while still permitting multi-DOF movement of the end effector, including wrist structure if any; be sufficiently thin to not add significantly to the overall outer dimensions of the instrument; and also be sufficiently durable to resist tearing or other damage to the sheath. In an exemplary embodiment, the sheath can be configured to provide effective electrical insulation for electrosurgical instruments that supply current to the body tissue under an applied voltage ranging from about 100 volts to 600 volts, for example for bipolar electrosurgical instruments, or for example as large as about 3000 volts for monopolar electrosurgical instruments, for example.

In at least some circumstances, an electrically insulative sheath, whether of a heat shrink tubing structure or other structure, provides sufficient protection against most electrical pathways that otherwise would potentially form along the electrically conductive components proximate the electrosurgical end effectors during an electrosurgical procedure. However, such a sheath may nevertheless provide interstitial openings between the sheath and the instrument that allow for the passage of blood and other liquids present in the wet environment associated with surgical procedures. This may allow liquids to enter interior regions of the surgical instrument, including at the proximal portions of the end effector, and consequently create pathways of lower electrical resistance with respect to pathway(s) between the end effector and the target tissue. In some cases, before performing an electrosurgical procedure on tissue, a surgeon may burn or evaporate off the liquid in the vicinity of the end effector by energizing the end effector. However, such a practice can be time-consuming and lead to dried fluid residue on the end effector structure, potentially resulting in lowered performance of the end effector to perform the desired tissue treatment. Further, in the case of bipolar electrosurgical end effectors, such unintended electrically conductive pathways can lead to short circuiting between proximal portions of the end effector structures that may be at differing electrical potentials. In the case of monopolar end effectors, sheaths in accordance with various exemplary embodiments also may provide protection against undesirable electrical pathways and also may provide sufficient flexibility to permit movement of such monopolar instrument end effectors, wrists, and/or portions of the shaft as noted above.

Thus, various exemplary embodiments contemplate a sheath assembly that includes an electrically insulative sheath and also an electrically insulative viscous material that at least partially fills the space between the sheath structure and the relevant electrosurgical instrument components covered by the sheath structure or at least partially fills a space within an electrosurgical instrument where an unintended electrical pathway could occur. In exemplary embodiments, the sheath is fit over the proximal portion of the end effector, any supporting structure that supports the end effector on the instrument shaft (e.g., clevis and/or wrist structure), and distal part of the shaft. The electrically insulative viscous material can serve as a barrier to liquids and other materials that may otherwise enter interstitial spaces of the end effector components via any openings provided between the sheath and the instrument when the sheath is disposed on the instrument.

In various exemplary embodiments, the viscous material may have a viscosity sufficient to ensure that once applied to the instrument, e.g., to at least partially fill the interstitial spaces of the proximal end portion of the end effector and/or between the sheath and the instrument, the material will remain substantially within the space protected by the sheath and not flow out. Further, aside from being electrically insulative and thus serving as an impediment to the creation of undesirable electrical pathways, the viscous material can serve as a barrier to prevent liquid and other unwanted materials from entering into the interstitial spaces of the end effector and associated components in any opening provided between the sheath and the instrument.

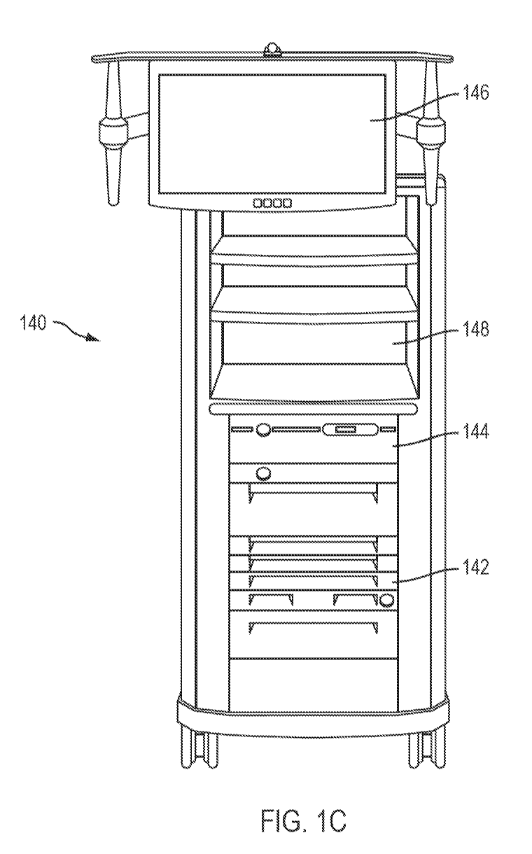

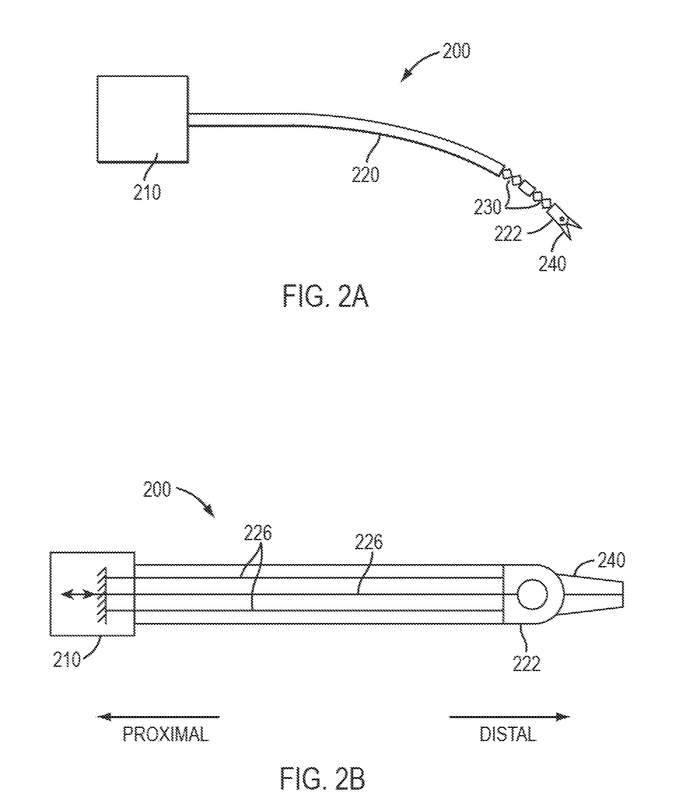

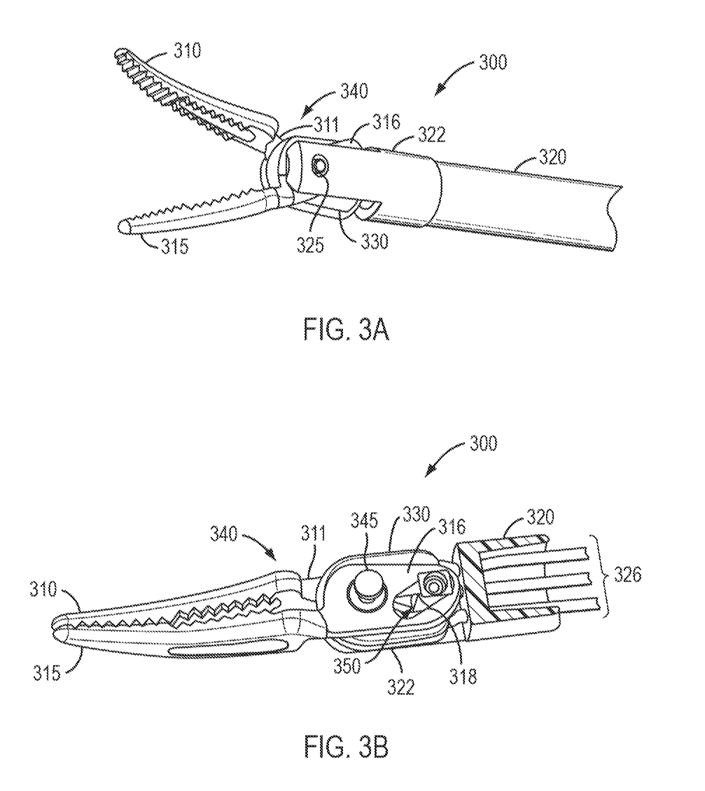

FIG. 3A shows a partial, perspective view of an exemplary embodiment of an end effector, clevis, and distal end portion of a main shaft of an electrosurgical instrument in accordance with an exemplary embodiment. As illustrated, end effector 300 can be a bipolar end effector that includes two jaw members 310, 315. Although the end effector 300 shown in FIGS. 3A-3C is a grasper, those having ordinary skill in the art would appreciate that the disclosure is not limited to such an embodiment and a variety of end effectors, including bipolar and/or monopolar, are within the scope of the present disclosure. Thus the end effector 300 can have a variety of other configurations, such as, for example, clamps, scissors, forceps scalpel, blade, hook, spatula, probe, needle point, dissectors, movable jaws (e.g., clamp), and any other type of surgical end effector equipment configured to manipulate and/or cauterize tissue and the like.

In the exemplary embodiment of FIGS. 3A-3C, the end effector is a bipolar end effector with one of the jaws 310, 315 connected to a positive electrode and the other of the jaws 310, 315 connected to a negative electrode. The jaws can be manipulated by pulling and/or pushing one or more force transmission mechanisms 326 actuated at a proximal end transmission housing (not shown), as those of ordinary skill in the art are familiar with. The jaws 310 and 315 have proximal extensions 311, 316 which are received within a clevis 322 that supports the end effector 300 to the instrument shaft 320. A clevis pin 345 can extend through holes 325 in distal ears of the clevis 322 and through holes in the jaw extensions 311, 316 to pivotably couple the jaws and clevis together, permitting the jaws to open and close as they pivot about the pin 345.

As shown in the exemplary embodiment of FIGS. 3A-3C, an electrical insulative septum 330 can be positioned between the extensions 311, 316 of the jaws 310, 315. The septum 330 can serve to provide some level of a barrier for the entry of liquid and other material proximally beyond the extensions 311, 316 and into other interior components of the instrument 300, as well as providing electrical insulation between the extensions 311, 316. In various exemplary embodiments, the septum may be made of various electrically insulative materials, including a plastic material, such as, for example, a PPA (polyphthalamide) plastic material. In various exemplary embodiments, suitable materials may also have a relatively high arc tracking index, which may help reduce the potential risk of any moisture wicking that would cause an arc to be sustained from one extension 311, 316 to another. One example of a suitable material for the septum 330 is a plastic of the trade name Amodel®.

When the jaws 310, 315 are open, no electrical pathway exists between the jaws 310, 315. When the jaws grasp or otherwise engage a tissue or a vessel, or other electrically conductive material positioned between the jaws 310, 315, an electrical current flows from one jaw to another and passes through the tissue, vessel, or other material. In the case of a tissue or vessel, the electrical current passing therethrough heats the vessel or tissue so as to seal or cut it depending on the electrocautery energy levels applied.

As shown in FIG. 3B, however, interstitial void spaces, such as shown at 350 for example, exist between portions within the clevis and between portions of the extensions 311, 316 of the jaws 310, 315. During operation, liquid and/or other conductive substances may accumulate in such interstitial spaces and cause short-circuiting between the jaws, particularly if the liquid and/or other substances accumulate proximate portions of the extensions 311, 316 where the septum 330 does not provide adequate insulative protection and separation of the extensions 311, 316. For example, the extensions 311, 316 can have cam slots (one of which is shown at 318 in FIG. 38) through which a pin moves during the opening and closing of the jaws 310, 315. In and around that area, the septum 330 may not provide sufficient protection to hinder electrical pathways formed between portions of the extensions 311, 316.

In accordance with an exemplary embodiment, FIG. 3C illustrates the instrument 300 equipped with an electrically insulative sheath 500 that is positioned to cover a distal end portion of the shaft 320, the clevis 322 and thus extensions 311, 316 housed in the clevis 322, and a proximal end portion of the jaws 310, 315. Those having ordinary skill in the art would appreciate that the sheath may have various lengths to cover various portions of an instrument. For instance, sheath 500 could extend over a greater portion and up to the entire length of the instrument shaft 320, and also cover a wrist or other supporting structure if present. The distal end of the sheath 500 is positioned to leave the main working portions of jaws 310, 315 uncovered and substantially unhindered in their opening and closing movements.

The sheath 500 is made of an electrically insulative material and positioned so that the proximal portions of the end effector 340 surrounded by the sheath 500, even if coming into contact with tissue or another instrument, will not form unintended electrical pathways. In various exemplary embodiments, the sheath 500 can be made of one or more materials arranged to provide the sheath with a dielectric strength ranging from 3000 V/mil to 5000 V/mil. As above, the sheath may be made of a material and configured to provide effective electrical insulation in applications of electrosurgical instruments having applied voltage ranges from about 100 volts to about 600 volts: however, it is contemplated that the sheath material may provide effective electrical insulations for applied voltage ranges as great as about 3000 volts.

The sheath 500 may be made of a single material or of composite layers of more than one material. Exemplary materials suitable for the sheath include, but are not limited to, polyester and fluoropolymers such as, for example, polytetrafluoroethylene (PTFE), poly(ethylene terephthalate) (PET), ethylene tetrafluoroethylene (ETFE), fluorinated ethylene-propylene FEP, perfluoroalkoxy polymer resin (PFA), and other organic materials that exhibit a relatively high dielectric strength. Reference is made to U.S. Patent App. Pub. No. US. 2012/0010611 A1 (published Jan. 12, 2012), entitled “Electrosurgical Tool Cover” to Krom et al., which is incorporated by reference herein, for various composite material layers for an electrosurgical instrument sheath that may be used in various exemplary sheath assemblies described herein.

The sheath 500 may be secured to the instrument so as to form a fluid-tight seal against the instrument, at least at the ends of the sheath. For example, the sheath may form a friction fit seal, a tension seal, or both.

In various exemplary embodiments, the sheath is made of a material and configured to be heat-shrinkable such that it can be heat shrunk to the instrument once put into the desired position relative thereto. Such a heat-shrunk sheath may result in a relatively tightly-fitting structure that conforms to the outer surface contours of the instrument, which can provide advantages relating to both minimizing the overall instrument size and not unduly restricting motion of the instrument.

In various exemplary embodiments, the wall thickness of the sheath 500 may range from about 0.0005 in. to about 0.05 in. For example for relatively small diameter instruments (e.g., of about 5 mm or less), the wall thickness may range from about 0.0005 in. to about 0.005 in., for example from about 0.001 in. to about 0.003 in., for example about 0.002 in., and have a dielectric strength of about 4000 Volts/mil to about 8000 Volts/mil. For larger diameter instruments, for example ranging from about 8 mm to about 15 mm, the wall thickness may range from about 0.005 in. to about 0.05 in., for example. The wall thickness is not anticipated to reduce significantly upon heat shrinking, for example, less than or equal to about 20% thickness reduction.

In one exemplary embodiment, the sheath 500 can be made of PET tubing having a wall thickness of about 0.002 inches and a dielectric strength ranging from 4000 Volts/mil to 8000 Volts/mil.

In order to satisfy the various desired uses of the sheath, the sheath can have a composite structure. As various sections of the sheath may be exposed in different work environments, the sheath may be made of different materials. For example, it can have a layered structure that includes different materials in different layers. By way of nonlimiting example, it may be desirable for the section of the sheath positioned around a wrist or other structure having a wide range of motion to exhibit a relatively high degree of elasticity to accommodate the movement of such structures, including, for example, accommodating mechanical joint motion of such structures. Further, it may be desirable to have the section of the sheath at or close to the work site (e.g., covering the part of the end effector) exhibit relatively high temperature and moisture resistance. Those of ordinary skill in the art would appreciate a variety of differing properties of the sheath that may be desirable for differing regions of the sheath depending, for example, on anticipated use and application of the sheath; accordingly, those of ordinary skill in the art would understand how to choose a variety of materials and configurations (e.g., layers, wall thickness, etc.) for the sheath based on various design considerations without departing from the scope of the present disclosure and claims.

Other sheath designs are applicable for use with the sheath assembly for electrosurgical instruments disclosed herein. For example, U.S. Patent App. Pub. No. US 2012/0010628 (published Jan. 12, 2012), entitled “Sheaths for Jointed Instruments,” to Cooper et al. and U.S. Patent App. Pub. No. US. 2012/0010611 A1 (published Jan. 12, 2012), entitled “Electrosurgical Tool Cover” to Krom et al., incorporated by reference herein, disclose various sheath configurations, all of which can be used in conjunction with the sheath assemblies for an electrosurgical instrument in accordance with the present disclosure.

Although the sheaths in accordance with various exemplary embodiments are configured to form friction and/or tension seals with the instrument and/or are heat shrunk to conform to the instrument, there may nonetheless exist openings between the sheath and the instrument through which liquid (e.g., blood, saline, etc.) and other materials can enter between the sheath and the instrument and potentially enter into interstitial spaces of the instrument itself, such as into proximal end portions of the end effector or supporting structures (such as, e.g., in spaces between wrist joints). As discussed above with reference to FIG. 3B, the space 350 is one example of an interstitial space that may be susceptible to the entry or accumulation of liquid, which can cause an electrical pathway between the jaws 310 and 320, in particular between the extensions 311 and 316, to be unintentionally formed leading to a short-circuit and/or to unintentional electrical pathways to the patient.

To protect against liquid entering into interstitial openings between a sheath and an electrosurgical instrument and thus into interstitial spaces of the instrument, an electrically insulative viscous material can be provided between the sheath and the instrument, according to an exemplary embodiment. For example, the electrically insulative viscous material can be applied around a supporting structure of the end effector, such as for example, the wrist structure 230, as well as to the proximal end portion of the end effector 340 corresponding to the extensions 311 and 316 and other portions received in the clevis 322. The electrically insulative viscous material can be applied before a sheath 500 is positioned on the instrument. Alternatively, the electrically insulative viscous material can be applied to the interior surfaces of the sheath 500 before the sheath is positioned on the instrument. In either case, the electrically insulative viscous material can work its way into the various interstitial spaces of the surgical instrument and end effector, and between the surgical instrument and the sheath. In exemplary embodiments, the electrically insulative viscous material also may be applied around one or both ends of the sheath to cover the junction where the sheath end meets the instrument.

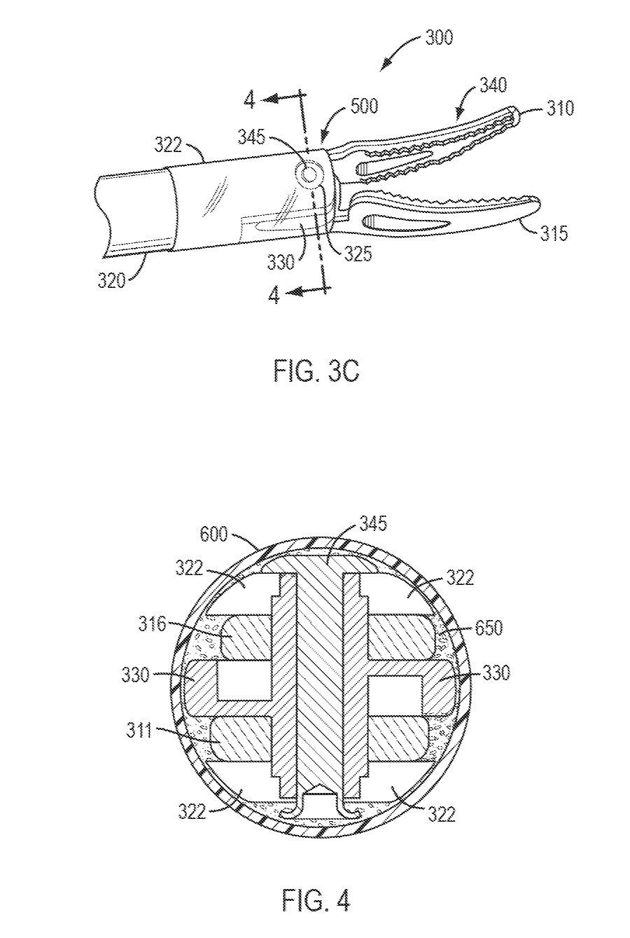

FIG. 4 illustrates a cross-section taken at line 4-4 of FIG. 3C depicting a sheath assembly including a sheath 600 and electrically insulative viscous material 650 installed on a surgical instrument 300 having the various end effector and supporting structure components as the embodiment of FIGS. 3A-3C. The sheath 600 can be configured as any of the electrically insulative sheath embodiments described herein. In embodiments wherein the sheath 600 is heat-shrinkable, the electrically insulative viscous material 650 can be applied first to either the instrument and/or the interior surfaces of the sheath before heat shrinking. Then, the sheath can be positioned on the instrument and heat can be applied thereto to shrink the sheath to secure it to the instrument. In one exemplary embodiment, a conventional heat gun used for heat shrink applications can be used at a temperature of about 300° C. to supply the heat for heat shrinking the sheath.

In exemplary embodiments that employ a wrist structure, such as, e.g., wrist structure 230 of FIG. 2A, the electrically insulative viscous material can be applied to fill interstitial spaces between joints of the wrist structure and in locations where the end effector and/or other components meet the wrist structure.

In yet another exemplary embodiment, the electrically insulative viscous material may be applied in an amount and location sufficient to form a barrier at the edges of the opposite ends of the sheath on the instrument. In either case, the electrically insulative viscous material can be disposed to protect against the entry of liquids and other materials underneath the sheath between the sheath and the electrosurgical instrument.

In various exemplary embodiments, the electrically insulative viscous material 650 can be a medically safe, substantially non-conductive lubricant. By way of example, the electrically insulative viscous material can exhibit electrical conductivity resistance of about 100 Ohms or more. The viscosity of the electrically insulative viscous material can be sufficiently high to prevent it from leaking out from between the sheath and the surgical instrument. For example, the dynamic viscosity of the electrically insulative viscous material may range from about 10 Pascal-seconds (Pa-s) to about 500 Pa-s, for example from about 50 Pa-s to about 300 Pa-s. In various exemplary embodiments, the viscosity may be such that the insulative viscous material is similar to viscous substances ranging from molasses to peanut butter, for example. Further, in various exemplary embodiments, the electrically insulative viscous material can exhibit heat resistance so it does not degrade in high temperature and substantially maintains its viscosity at higher temperatures.

One example of a suitable electrically insulative viscous material that can be used with the sheath assemblies of the present disclosure is an insulative grease comprising perfluoropolyether-based oil and polytetrafluoroethylene powder, such as Krytox® grease made by DuPont™. For example, Krytox® GPL206 may be used. Other suitable electrically insulative viscous materials include dielectric silicone greases.

Various exemplary embodiments of the present disclosure contemplate a kit comprising an electrically insulative sheath configured to be positioned on a surgical instrument to surround at least a proximal end of an end effector of the surgical instrument; and an electrically insulative viscous material, wherein the electrically insulative material has a viscosity sufficient to hold the material within an interior region defined by the sheath.

Further, various exemplary embodiments contemplate a method that includes applying an electrically insulative viscous material to one of a portion of an electrosurgical instrument that includes at least a proximal end region of an end effector of the electrosurgical instrument and an interior surface portion of a sheath, positioning a sheath on the electrosurgical instrument such that the electrically insulative viscous material is within an interior region defined by the sheath, and heat shrinking the sheath positioned on the electrosurgical instrument.

The method can further include applying the electrically insulative viscous material to the portion of the electrosurgical instrument before positioning the sheath, such as to the interior surface portion of the sheath before positioning the sheath and/or around at least one end of the sheath at a junction of the sheath end and the instrument.

Further modifications and alternative embodiments will be apparent to those of ordinary skill in the art in view of the disclosure herein. For example, the systems and the methods may include additional components or steps that were omitted from the diagrams and description for clarity of operation. Accordingly, this description is to be construed as illustrative only and is for the purpose of teaching those skilled in the art the general manner of carrying out the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the all after having the benefit of the description herein. Changes may be made in the elements described herein without departing from the spirit and scope of the present teachings and following claims.

It is to be understood that the particular examples and embodiments set forth herein are nonlimiting, and modifications to structure, dimensions, materials, and methodologies may be made without departing from the scope of the present teachings. For example, various aspects have been described in the context of an instrument used in a teleoperated surgical system. But these aspects may be incorporated into hand-held, manually operated instruments as well. Further, the illustrated embodiments have been described with reference to a bipolar electrosurgical instrument, however the exemplary sheaths and sheath assemblies described can be used in conjunction with monopolar electrosurgical instruments and systems as well based on modification within the level of ordinary skill in the art.

Exemplary embodiments of the present disclosure have been described in detail. Other embodiments will become apparent to those skilled in the all from consideration and practice of the present disclosure. Accordingly, it is intended that the specification and the drawings be considered as exemplary and explanatory only, with the claims being entitled to their full scope and breadth, including equivalents.

What is claimed is:

1. An electrosurgical device, comprising:
an elongated shaft having a distal end and a proximal end;
an electrosurgical end effector coupled to the distal end of the elongated shaft;
an electrically insulative sheath disposed around at least a proximal end portion of the end effector; and
an electrically insulative viscous material disposed to provide a barrier to liquid entry into an interior region defined by the sheath.

2. The electrosurgical device of claim 1, wherein the electrically insulative viscous material is disposed at least at the distal end of the sheath between the sheath and the instrument.

3. The electrosurgical device of claim 1, wherein the electrically insulative viscous material has a viscosity sufficient to prevent the electrically insulative viscous material from flowing out of the sheath between the instrument and the sheath.

4. The electrosurgical device of claim 3, wherein the electrically insulative viscous material has a dynamic viscosity ranging from 10 Pa-s to-500 Pa-s.

5. The electrosurgical device of claim 1, wherein the electrically insulative viscous material comprises an electrically insulative grease.

6. The electrosurgical device of claim 1, wherein the electrically insulative viscous material comprises perfluoropolyether-based oil and polytetrafluoroethylene powder.

7. The electrosurgical device of claim 1, wherein the electrosurgical end effector comprises a bipolar energy electrosurgical end effector.

8. The electrosurgical device of claim 1, wherein the end effector is configured to perform at least one surgical procedure chosen from tissue cutting, tissue grasping, tissue sealing, and tissue ablation.

9. The electrosurgical device of claim 1, further comprising a support structure that couples the end effector to the distal end of the elongated shaft, wherein the sheath is disposed to surround the support structure.

10. The electrosurgical device of claim 9, wherein the support structure comprises a wrist structure.

11. The electrosurgical device of claim 1, wherein the sheath is made of a material is chosen from polyester, polytetrafluoroethylene, ethylene tetrafluoroethylene, fluorinated ethylene-propylene, and pertluoroalkoxy polymer resin.

12. The electrosurgical device of claim 1, wherein the sheath is made of a heat-shrinkable material and is secured by heat shrinking to the instrument.

13. The electrosurgical device of claim 1, wherein the sheath has a wall thickness ranging from 0.0005 to 0.005 inches.

14. The electrosurgical device of claim 1, wherein the sheath has a dielectric strength ranging from 3000 Volts/mil to 8000 Volts/mil.

15. The electrosurgical device of claim 1, wherein the electrosurgical instrument is configured for use in a teleoperated surgical system.

16. The electrosurgical device of claim 1, wherein the electrically insulative viscous material is disposed to at least partially fill an interstitial space in one or more components of the end effector.

17. The electrosurgical device of claim 1, wherein the electrically insulative viscous material is disposed in an amount and arrangement sufficient to protect against an unintended electrical pathway formed at least in part by a component of the electrosurgical instrument.

18. A sheath assembly for an electrosurgical instrument, the sheath assembly comprising:
an electrically insulative sheath configured to be positioned on a surgical instrument to surround at least a proximal end of an end effector of the surgical instrument; and
an electrically insulative viscous material disposed within an interior region defined by the sheath.

19. The sheath assembly of claim 18, wherein the electrically insulative viscous material has a viscosity sufficient to substantially prevent the electrically insulative viscous material from flowing outside of the interior region.

20. The sheath assembly of claim 19, wherein the electrically insulative viscous material has a dynamic viscosity ranging from 10 Pa-s to 500 Pa-s.

21. The sheath assembly of claim 18, wherein the electrically insulative viscous material comprises a perfluoropolyether-based oil and a polytetrafluoroethylene powder.

22. The sheath assembly of claim 18, wherein the electrically insulative sheath comprises a material chosen from polyester, polytetrafluoroethylene, ethylene tetrafluoroethylene, fluorinated ethylene-propylene, and perfluoroalkoxy polymer resin.

23. An electrosurgical device comprising:
an electrosurgical instrument comprising an elongated shaft having a distal end and a proximal end, and an electrosurgical end effector coupled to the distal end of the elongated shaft; and
an electrically insulative viscous material disposed in an amount and arrangement sufficient to protect against an unintended electrical pathway formed at least in part by a component of the electrosurgical instrument.

24. The electrosurgical device of claim 23, wherein the electrically insulative viscous material has a dynamic viscosity ranging from 10 Pa-s to 500 Pa-s.

25. The electrosurgical device of claim 23, wherein the electrically insulative viscous material is disposed to at least partially fill an interstitial space
